(19) United States
(12) Patent Application Publication
(10) Pub. No.: US 2024/0417679 A1
(43) Pub. Date: Dec. 19, 2024

(54) METHOD FOR PRODUCING ASYMMETRIC HAIR FOLLICLE SPHEROID, AND ASYMMETRIC HAIR FOLLICLE SPHEROID PRODUCED BY SAME METHOD

(71) Applicant: T&R BIOFAB CO., LTD., Siheung-si (KR)

(72) Inventors: Songwan Jin, Siheung-si (KR); Jin-Hyung Shim, Siheung-si (KR); Won-Soo Yun, Siheung-si (KR); Paulo Andre Nobrega Marinho, Seongnam-si (KR); Ilho Jang, Gwangju-si (KR); Daesik Kim, Yongin-si (KR)

(73) Assignee: T&R BIOFAB CO., LTD., Siheung-si (KR)

(21) Appl. No.: 18/821,020

(22) Filed: Aug. 30, 2024

Related U.S. Application Data
(63) Continuation of application No. PCT/KR2023/001332, filed on Jan. 30, 2023.

(30) Foreign Application Priority Data
Mar. 4, 2022 (KR) 10-2022-0028006

(51) Int. Cl. C12N 5/00; C12N 5/071

(52) U.S. Cl. CPC C12N 5/0062; C12N 5/0627; C12N 5/069; C12N 2513/00

(57) ABSTRACT

The present invention relates to a method for producing an asymmetric hair follicle spheroid comprising: an accommodating part preparation step of preparing a bio-ink accommodating part in which a partitioning member providing a plurality of partitioned spaces is accommodated; a bio-ink supplying step of positioning the partitioning member in the bio-ink accommodating part and supplying bio-ink containing hydrogel to each partitioned space of the partitioning member; and a discharge step of applying pressure to the bio-ink accommodating part and discharging the bio-ink accommodated in the partitioned spaces through a single nozzle. The asymmetric hair follicle spheroid produced according to the present disclosure allows the direction of hair growth to be controlled.

### CROSS-REFERENCE TO RELATED PATENT APPLICATION

This application is a continuation of international application PCT/KR2023/001332, filed on Jan. 30, 2023, which claims the benefit of Korean Patent Application No. 10-2022-0028006, filed on Mar. 4, 2022, in the Korean Intellectual Property Office, the disclosure of which is incorporated herein in its entirety by reference.

### TECHNICAL FIELD

The present disclosure relates to a method for producing an asymmetric hair follicle spheroid and an asymmetric hair follicle spheroid produced by the same method, and more particularly, to a method for producing an asymmetric hair follicle spheroid in which cells are distributed in an asymmetric form so as to provide an effect that hair grows in a specific direction by filling a plurality of bio-inks, each of which includes outer root sheath cells, dermal papilla cells, and inner root sheath cells into a partitioning member whose interior is partitioned into a plurality of regions in an asymmetric shape, respectively, and then performing a printing process of simultaneously discharging the bio-inks, and an asymmetric hair follicle spheroid produced by the same method.

### BACKGROUND ART

Human hair is very important not only because it plays a primary role in protecting the skin and scalp, but also because it plays a unique role in social and sexual communications. Therefore, research into hair and hair loss has been continuously conducted, but specific mechanisms related to this have not yet been clearly identified.

Hair is composed of keratin protein, and is produced and grows from hair follicles in the dermis. It has been known that Scalp hair follicles are composed of dermal papilla cells, keratinocytes, inner root sheath cells, outer root sheath cells, melanocytes, etc., and among these, dermal papilla cells are the basis of hair follicle development and are closely related to hair growth.

Dermal papilla cells are cells located in dermal papilla at the base of the hair follicle. Dermal papilla cells have capillaries distributed through them, which supply nutrients to the hair follicle and secrete growth factor and inhibition factor to control the growth of hair follicle epithelial cells.

Cell division and migration near the dermal papilla are closely related to hair growth, and when new hair is produced from the dermal papilla during a growth phase, cells are activated by various cytokines, hormones, etc., and cell division and migration occur, which affects hair growth.

Hair growth is regulated by an action of various factors around the hair follicle, an interaction between epidermal cells and dermal papilla cells in the hair follicle, etc. During a growth phase when hair is actively grows, active proliferation and differentiation of dermal papilla cells occurs, while during a catagen phase, a resting phase, and a hair loss phase when hair growth stops, dermal papilla cells died.

Currently, in order to study a method for treating hair loss or growth of hair follicle cells, a technology for culturing follicle spheroids in vivo has been studied. In general, methods such as hanging drop, spontaneous spheroid formation, suspension culture, scaffold based models, and magnetic levitation are applied to produce spheroids. However, when two or more types of cells, namely outer root sheath cells, inner root sheath cells, and dermal papilla cells, are co-cultured with hair follicle spheroids, it is difficult to produce hair follicle spheroids by applying these methods, and the cells need to be clustered sequentially, which is time-consuming, and a size deviation of each cell spheroid is large.

In addition, hair follicles and hairs growing in the body are naturally oriented, but hair follicle spheroids in vitro have difficulty in imparting directionality to hair growing in the hair follicle, which is a major obstacle in producing hair follicle spheroids with uniform quality. Therefore, it is necessary to develop a technology that may give directionality to hair follicles and hair growth when producing hair follicle spheroids in vitro, and that may form the quality of individual hair follicle spheroids uniformly.

### DISCLOSURE

### Technical Problem

An aspect of the present disclosure is to provide a method for producing an asymmetric hair follicle spheroid capable of providing an effect that hair grows in a specific direction by filling a plurality of bio-inks, each of which includes outer root sheath cells, dermal papilla cells, and inner root sheath cells into a partitioning member having regions partitioned in an asymmetric shape, respectively, and performing printing, and an asymmetric hair follicle spheroid produced by the same method.

### Technical Solution

In an aspect, there is provided a method for producing an asymmetric hair follicle spheroid including: an accommodating part preparation step of preparing a bio-ink accommodating part 130; a loading step of positioning a partitioning member 110 in which a plurality of partitioned spaces are formed, in the bio-ink accommodating part 130; a bio-ink supplying step of supplying bio-ink containing hydrogel to each partitioned space of the partitioning member 110; and a discharge step of applying pressure to the bio-ink accommodating part 130 and discharging the bio-ink accommodated in the partitioned space through a single nozzle.

A cross section of the partitioning member 110 in which the plurality of partitioned spaces are formed, has a structure in which it is asymmetric with respect to an X-axis and is symmetric with respect to a Y-axis.

The partitioning member 110 includes the plurality of partitioned spaces including: a first partitioned space 111 extending from a lower portion of the partitioning member 110 to a middle upper portion of the partitioning member 110 along the Y-axis of the partitioning member 110; a second partitioned space 112 formed on an upper portion of the partitioning member 110 so as to surround the upper portion of the first partitioned space 111 and having a half-donut shape; and a plurality of third partitioned spaces 113 having one side surface formed by the first partitioned space 111 and an upper surface formed by the second partitioned space 112.

First bio-ink containing dermal papilla cells may be supplied to the first partitioned space 111, second bio-ink containing outer root sheath cells may be supplied to the second partitioned space 112, and third bio-ink containing vascular endothelial cells may be supplied to the third second partitioned space 113.

The discharge step may be performed simultaneously with the bio-ink supplying step or may be performed sequentially after the bio-ink supplying step.

In another aspect, there is provided an asymmetric hair follicle spheroid produced by the method described above and having a spherical shape or a shape similar to the spherical shape.

A cross section of the hair follicle spheroid is asymmetric with respect to an X-axis and is symmetric with respect to a Y-axis.

The cross section of the hair follicle spheroid includes: a first region 210 formed to extend from a lower portion of the hair follicle spheroid to a central portion of the hair follicle spheroid along the Y-axis; a second region 220 having a half-donut shape and having a concave portion formed to surround an upper portion of the first region 210; and a third region 230 formed to surround the remaining side surfaces of the first region 210.

### Advantageous Effects

The asymmetric hair follicle spheroid produced according to a method for producing an asymmetric hair follicle spheroid of the present disclosure may impart directionality to hair follicle and hair growth.

In addition, individual hair follicle spheroids are produced by a bio-printing method, such that the individual hair follicle spheroids are simply and fast produced, and have a uniform quality.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic diagram illustrating a part of a method for producing an asymmetric hair follicle spheroid according to an embodiment of the present disclosure.

FIG. 2 is a diagram illustrating a cross section of a partitioning member in more detail.

FIGS. 3A to 3D are diagrams illustrating cross-sectional shapes of a partitioning member.

FIG. 4 is a schematic diagram illustrating a general hair production process.

FIG. 5 is a schematic diagram illustrating preset extrusion method using a micro-fluidic device of the present disclosure.

FIG. 6 is a schematic diagram illustrating a cross-sectional shape of an asymmetric hair follicle spheroid according to another embodiment of the present disclosure.

FIG. 7 illustrates a fluorescence observation result of a hair follicle spheroid produced in Preparation Example 1.

FIG. 8 is a diagram illustrating the results of Experimental Examples.

[Description of Reference Numerals]

| | |
|---|---|
| 110: Partitioning member | 111: First partitioned space |
| 112: Second partitioned space | 113: Third partitioned space |
| 114: Outer wall | 130: Bio-ink accommodating part |
| 140: Co-axial nozzle | 161: Inner nozzle |
| 170: Outer nozzle | 200: Asymmetric hair follicle spheroid |

### BEST MODE FOR INVENTION

Before describing the present disclosure in detail through preferred embodiments of the present disclosure below, it is to be understood that terms or words used in the present specification and claims should not be interpreted as limited to their usual or dictionary meanings, but should be interpreted as meanings and concepts that conform to the technical idea of the present disclosure.

Throughout the present specification, “including” any component will be understood to imply the inclusion of other components rather than the exclusion of other components, unless specifically stated to the contrary.

Throughout the present specification, “%” used to indicate the concentration of a specific substance refers to (weight/weight) % for solid/solid, (weight/volume) % for solid/liquid, and (volume/volume) % for liquid/liquid, unless otherwise stated.

The respective steps may be performed in an order different from the stated order unless the context clearly states a specific order. That is, the respective steps may be performed in the same order as the stated order, may be performed substantially simultaneously, or may be performed in the opposite order.

In the drawings, portions unrelated to the description will be omitted to clearly describe the disclosure proposed, and similar reference numerals will be used to describe similar portions throughout the specification. In addition, as used herein, a “part” refers to a unit or block that performs a specific function.

Hereinafter, an embodiment of the present disclosure will be described. However, the scope of the present disclosure is not limited to the following preferred embodiments, and those skilled in the art may implement various modified forms of the contents described in this specification within the scope of the present disclosure.

The present disclosure relates to a method for producing an asymmetric hair follicle spheroid and an asymmetric hair follicle spheroid produced by the same method, and more particularly, to a method for producing an asymmetric hair follicle spheroid capable of providing an effect that hair grows in a specific direction by filling a plurality of bio-inks, each of which includes outer root sheath cells, dermal papilla cells, and inner root sheath cells into a partitioning member whose interior is partitioned into an asymmetric shape, respectively, and performing printing, and an asymmetric hair follicle spheroid produced by the same method.

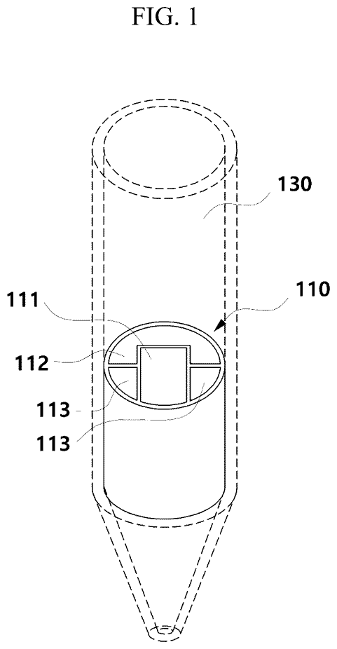

First, FIG. 1 is illustrated to describe a method for preparing an asymmetric hair follicle spheroid according to an embodiment of the present disclosure, and hereinafter, it will be described with reference to FIG. 1.

A method for producing an asymmetric hair follicle spheroid according to an embodiment of the present disclosure includes: an accommodating part preparation step of preparing a bio-ink accommodating part 130; a loading step of positioning a partitioning member 110 in which a plurality of partitioned spaces are formed, in the bio-ink accommodating part 130; a bio-ink supplying step of supplying bio-ink containing hydrogel to each partitioned space of the partitioning member 110; and a discharge step of applying pressure to the bio-ink accommodating part 130 and discharging the bio-ink accommodated in the partitioned spaces through a single nozzle.

According to an embodiment of the present disclosure, an asymmetric hair follicle spheroid is formed by supplying three or more of bio-inks to different partitioned spaces and applying pressure to discharge the bio-inks. Here, when the partitioned spaces to which the bio-inks are supplied are disposed in a predetermined form, directionality is given to the hair that is produced and grows from the hair follicle spheroid, so that it can be applied more easily for research and practical purposes.

The partitioning member 110 in the accommodating part preparation step has a special structure for producing a hair follicle spheroid capable of giving directionality to hair.

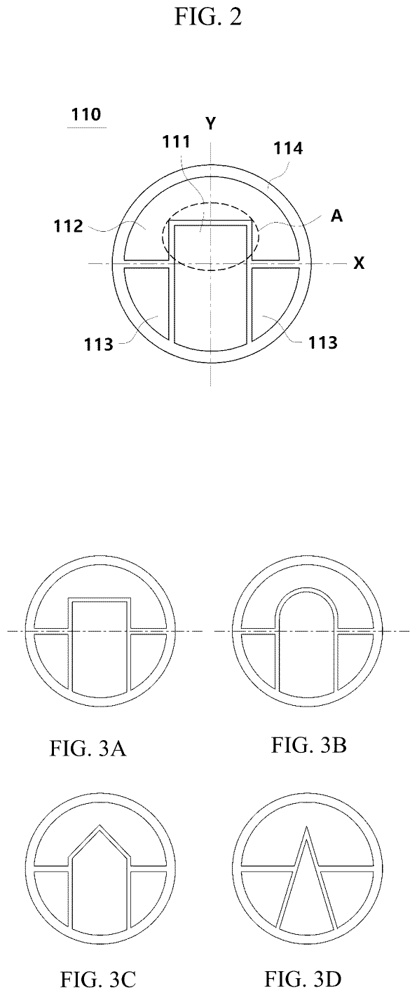

FIG. 2 is a diagram illustrating the partitioning member 110 in more detail, wherein the partitioning member 110 has a tubular shape whose interior is partitioned into a plurality of partitioned spaces, and a cross section of the partitioning member 110 has a shape in which it is asymmetric with respect to an X-axis that is a horizontal axis, and is symmetric with respect to a Y-axis that is a vertical axis. Here, the X-axis and the Y-axis refers to axes that are orthogonal to each other in the cross section of the partitioning member 110, and an intersection point where the X-axis and the Y-axis intersect may be a center point of the partitioning member 110.

The partitioning member 110 is partitioned into a plurality of partitioned spaces including a first partitioned space 111, a second partitioned space 112, and a third partitioned space 113.

The first partitioned space 111 is formed to extend from a lower part of the partitioning member 110 to a middle upper portion (herein, ‘middle upper portion’ refers to any position between the center and the top) of the partitioning member 110 along the Y-axis of the partitioning member 110. The second partitioned space 112 is formed on an upper portion of the partitioning member 110 so as to surround an upper portion A of the first partitioned space 111 and has a half-donut shape. The third partitioned space 113 has one side surface formed by the first partitioned space 111 (i.e., one side surface is in contact with the first partitioned space 111, and the other side surface is in contact with an outer periphery of the partitioning member 110), and an upper surface formed by the second partitioned space 112 (i.e., the upper surface is in contact with the second partitioned space 112).

As used herein, the term “half-donut shape” refers to a shape in which a ring structure such as a tire tube or a donut is cut in half, and a shape of a line forming an inner hole may have an arc shape, and may also have a shape of a polygonal straight line with at least one vertex.

An upper surface or an upper point of the first partitioned space 111 is located above the X-axis that passes horizontally through the center of the cross section, and an upper surface of the third partitioned space 113, that is, a lower surface of the second partitioned space 112, is formed so that it is located on the same line as the X-axis or on a line close to the X-axis.

Accordingly, the first partitioned space 111 and the third partitioned space 113 have a convex shape in which a convex portion is formed, and the second partitioned space 112 has a concave shape in which a concave portion is formed, and the first, second, and third partitioned spaces 111, 112, and 113 are respectively disposed inside the partitioning member 110 so that the convex portion having convex shape is surrounded by the concave portion having concave shape.

An outer wall 114 of the partitioning member 110 may be formed to have a thickness in the range of 0.8 to 1.5 mm. When the outer wall 114 is formed in such a thickness range, a velocity difference a central portion of the partition member 110 and a peripheral portion region in contact with the outer wall 114 is reduced when a fluid flows, such that the bio-ink may be uniformly discharged over an entire internal region of the partition member 110.

A partitioning wall defining each partitioned space may be formed to have a thickness of 0.1 to 0.3 mm, preferably 0.12 to 0.19 mm. when the partitioning wall is formed in such a thickness range, a cross-sectional shape of the hair follicle spheroid is formed more similarly to a cross-sectional shape of the partitioning member 110. Therefore, the shape of the hair follicle spheroid may be easily controlled, and the hair follicle spheroid with more uniform quality may be produced.

In addition, in order to secure the directionality of the first bio-ink discharged from the first partitioned space 111, it is preferable that a width of the first partitioned space 111 on the X-axis is wider than a width of each of the third partitioned space 113. This is because when the width of the third partitioned space 113 is formed wider than the width of the first partitioned space 111, the first bio-ink may not form a single stream or line in the hair follicle spheroid, and may be embedded in or mixed with the third bio-ink.

In FIG. 2, the upper portion A of the first partitioned space is illustrated to be a square, but as illustrated n FIGS. 3A to 3D which illustrate various cross-sectional shapes of the partitioning member 110, the upper portion A of the first partitioned space may be a polygon such as a semicircle, a triangle, or a pentagon.

The bio-ink supplying step is a step of supplying bio-ink containing hydrogel to each partitioned space of the partitioning member 110. Here, bio-inks of different compositions may be supplied to some of the partitioned spaces of each partitioned space, so that a hair follicle spheroid having an asymmetric structure therein may be formed.

Specifically, the first bio-ink may be supplied to the first partitioned space 111, the second bio-ink may be supplied to the second partitioned space 112, and the third bio-ink may be supplied to the third partitioned space 113.

The first bio-ink, the second bio-ink, and the third bio-ink contain hydrogels, and may contain the same hydrogel or may also contain different hydrogels.

The hydrogel may contain one or more selected from the group consisting of collagen, agarose, alginate, fibrinogen, fibrin, laminin, carboxymethyl cellulose, heparin sulfate, hyaluronic acid, dextran, gelatin, pluronic, polyoxyethylene-polyoxypropylene block copolymer, silicone, polysaccharide, polyethylene glycol, polyurethane, bio-ink decellularized from a specific tissue, matrigel, and hydroxyapatite.

The hydrogel may have a pH of 7 to 8, preferably 7.1 to 7.5, and such a pH range of the hydrogel is similar to that of the human body which may increase cell viability in the spheroid.

Each bio-ink may contain different biological cells to form a hair follicle spheroid having an asymmetric structure. Specifically, the first bio-ink may contain dermal papilla (DP) cells, the second bio-ink may contain outer root sheath (ORS) cells, and the third bio-ink may contain vascular endothelial (Endo) cells.

Biological cells contained in each bio-ink may be contained at a concentration of 10⁴ cells/ml to 10⁹ cells/ml. When these biological cells are contained in the hair follicle spheroid at an appropriate level as described above, biological activity may be optimized.

When the biological cells described above are contained in each bio-ink to form a hair follicle spheroid having an asymmetric structure, a hair follicle spheroid may be formed in which an upper portion of first bio-ink containing the dermal papilla cells is surrounded by second bio-ink containing the outer root sheath cells, and at the same time, and third bio-ink containing vascular endothelial cells is disposed on both sides of the first bio-ink.

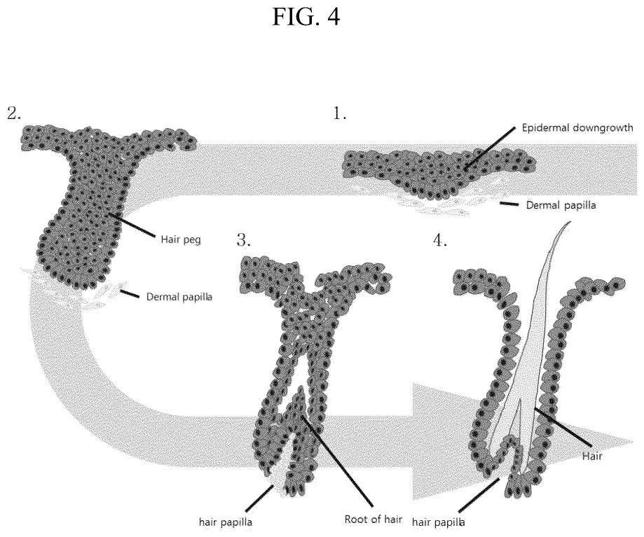

If the viscosity is too low, the hair follicle spheroids discharged from a nozzle do not maintain a spherical shape or the compartments are mixed with each other. If the viscosity is too high, the shear force and viscosity become excessively large, so that the spherical shape cannot be maintained when discharged, a certain amount of bio-ink cannot be discharged from each partitioned space, excessive force is required for discharge, or discharge does not occur. Therefore, it is preferable that the bio-ink have a viscosity within the above-described range. FIG. 4 is a schematic diagram illustrating a general hair production process, wherein hair formed by cell division of the outer root cells in hair pillar is produced in the direction of the hair root, but when a hair follicle spheroid is formed by a method according to an embodiment of the present disclosure, hair may be produced from the first bio-ink containing the dermal papilla cells in the direction of the second bio-ink containing the outer root sheath cells, thereby controlling the direction of hair production.

In addition, when the hair follicle spheroid includes dermal papilla cells, outer root sheath cells, and vascular endothelial cells, and these biological cells are disposed in the form described above and co-cultured, the direction of hair production may be controlled, hair production may be significantly promoted, and good growth factor necessary for hair follicle or hair formation may be supplied to the hair follicle spheroid due to vascular endothelial cells.

The discharge step is a step of applying pressure to the bio-ink accommodating part 130 and discharging the bio-ink accommodated in the partitioned spaces through a single nozzle. This step may be performed simultaneously with the bio-ink supplying step, or may be performed sequentially after the bio-ink supplying step.

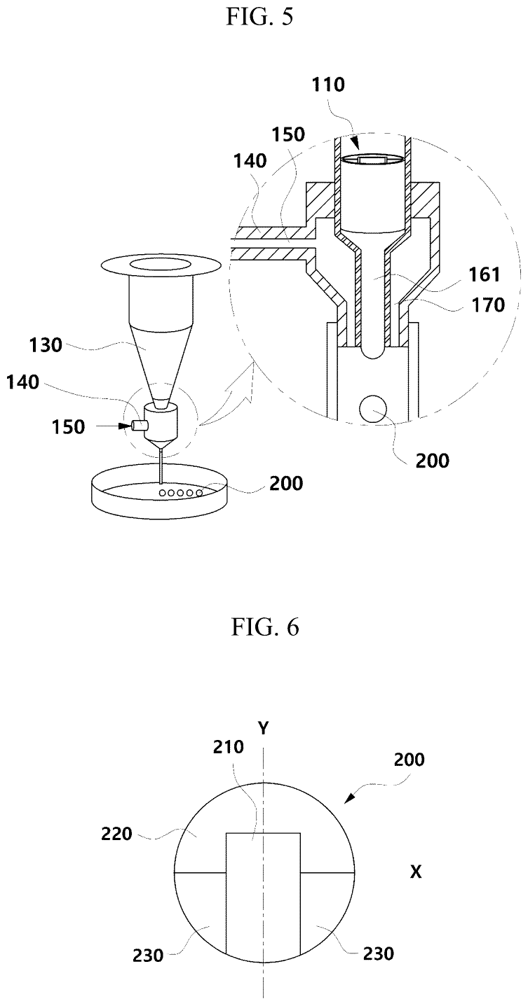

In the discharge step, the bio-ink accommodated in the partitioned space may be discharged using a preset extrusion method using a micro-fluidic device to form a hair follicle spheroid. A preset extrusion method using such a micro-fluidic device is schematically illustrated in FIG. 5.

When the preset extrusion method using the micro-fluidic device is used, the discharge step may be performed using a micro-fluidic device configured in a manner in which a lower discharge part of the bio-ink accommodating part 130 is formed in the form of a co-axial nozzle 140 or a separate co-axial nozzle 140 is coupled to the lower discharge part of the bio-ink accommodating part 130.

Using the co-axial nozzle 140, a hydrophobic solution 150 may be continuously discharged through an outer nozzle 170 and the bio-ink may be intermittently discharged through an inner nozzle 161 to form a spherical hair follicle spheroid. In this case, the nozzle through which the first, second, and third bio-inks forming the hair follicle spheroid are discharged is the inner nozzle 161 of the co-axial nozzle.

In such a case, the discharge step includes: continuously discharging a hydrophobic solution 150 through an outer nozzle 170 of a co-axial nozzle; and intermittently applying pressure to an inner nozzle 161 of a co-axial nozzle and discontinuously discharging first, second, and third bio-inks filled in a partitioning member 110.

As described above, the discharge step may be performed simultaneously with the bio-ink supplying step or may be performed after the bio-ink supplying step.

For example, when the bio-ink supplying step and the discharge step are performed simultaneously, the first bio-ink may be supplied to the first partitioned space 111, the second bio-ink may be supplied to the second partitioned space 112, and the third bio-ink may be supplied to the third partitioned space 113 while pressure may be applied intermittently to the bio-ink accommodating part 130 so that the first, second, and third bio-inks may be discharged together.

As another example, when the discharge step is performed after the bio-ink supplying step, the partitioning member 110 in which the first partitioned space 111 is filled with the first bio-ink, the second partitioned space 112 is filled with the second bio-ink, and the third partitioned space 113 is filled with the third bio-ink, may be positioned in the bio-ink accommodating part 130 and pressure may be applied to the inner nozzle 161 to discharge the first, second, and third bio-inks together.

When the first, second and third bio-inks filled in the first, second and third partitioned spaces 111, 112, and 113 in the partitioning member 110 are discharged at the same time, as illustrated in FIG. 6, a hair follicle spheroid is formed in which an upper portion of the first bio-ink is surrounded by the second bio-ink, and the third bio-ink is disposed on both sides of the first bio-ink.

In this case, in order to enable the discharged bio-ink to be discharged intermittently rather than continuously, it is preferable that a nozzle end of the bio-ink accommodating part 130 be configured in the form of a co-axial nozzle 140.

By using the co-axial nozzle 140 including an inner nozzle 161 and an outer nozzle 170, a hair follicle spheroid having an outer peripheral surface with a spherical shape or a round shape close to the spherical shape may be formed while the hydrophobic solution 150 is discharged in a continuous phase through the outer nozzle 170 and the hydrophilic solution is discharged in a dispersed phase through the inner nozzle 161.

Here, the phrase “discharged in a continuous phase” means that the solution is continuously discharged, and the phrase “discharged in a dispersed phase” means that the solution is intermittently discharged.

That is, cell aggregates or cell micro-tissues having an outer peripheral surface with a relatively uniformly size and a spherical shape or a round shape close to the spherical shape may be formed by discharging the hydrophobic solution in the continuous phase through the outer nozzle 170 and the hydrophilic solution in the dispersed phase through the inner nozzle 161 using the immiscibility of the hydrophobic solution and the hydrophilic solution.

The hydrophobic solution 150 may contain an oil and a surfactant. According to a preferred embodiment, the oil may include one or more selected from the group consisting of mineral oil, synthetic oil, vegetable oil, and silicone oil, and the surfactant may include one or more selected from the group consisting of sorbitan monooleate (SPAN 80), cetyl PEG/PPG-10/1 dimethicone (ABIL EM-90), sorbitan sesquioleate (ARLACEL 83), polyethylene glycol, and dipolyhydroxy stearate (ARLACEL P135).

More preferably, the hydrophobic solution 150 may contain oil alone or may contain oil and a surfactant together. That is, the hydrophobic solution may be a mixture of oil and a surfactant in a weight ratio of 1:0 to 0.05. If the surfactant is excessively included beyond the above weight ratio, a discharge speed is degraded or the interaction with the hydrophilic solution is weakened, and thus it is difficult to form a spheroid having a spherical shape or a round shape, which is undesirable.

Meanwhile, according to an embodiment, the hydrophilic solution discharged through the inner nozzle 161 may include first, second, and third bio-inks that were prefilled through the partitioning member 110 described above.

A diameter or an effective diameter of the hair follicle spheroid may be in the range of about 50 to 1,000 μm, where the effective diameter means the diameter of a sphere converted from a surface area of a non-spherical structure.

The size of the diameter or the effective diameter may be determined by the diameter of the inner nozzle 16 and the diameter of the outer nozzle 170 in the co-axial nozzle 140, the flow rate, and the like. According to an embodiment, the inner diameter of the outer nozzle may be about 1.0 to 2.0 mm, an outer diameter of the inner nozzle may be about 0.5 to 1.0 mm, and an inner diameter of the inner nozzle may be about 0.3 to 0.7 mm. The ranges of the inner diameter of the outer nozzle, the outer diameter of the inner nozzle, and the inner diameter of the inner nozzle within such a range are ranges capable of more uniformly controlling the size of the discharged hair follicle spheroid.

In this case, preferably, the pressure applied to the outer nozzle 170 is in the range of about 10 to 150 kPa, and the pressure applied to the inner nozzle 161 is in the range of about 5 to 50 kPa. Such a discharge speed of the hydrophobic solution and the hydrophilic solution, and the pressure of the outer nozzle and the inner nozzle, are in the range of preferred conditions for forming uniform hair follicle spheroids having an effective diameter in the range of 50 to 1,000 μm.

Through these steps, the hair follicle spheroids discharged through the co-axial nozzle 140 may be cultured in a culture container for about 12 to 24 hours, and through this culture process, each hair follicle spheroid may produce hair in a direction from where the first bio-ink containing the dermal papilla cells is located to where the second bio-ink containing the outer root sheath cells is located.

Meanwhile, another embodiment of the present disclosure relates to an asymmetric hair follicle spheroid, and since the asymmetric hair follicle spheroid of the present embodiment may be produced through a method according to an embodiment of the present disclosure, the overlapping description will be omitted.

FIG. 6 is a schematic diagram illustrating a cross-sectional shape of an asymmetric hair follicle spheroid 200 according to another embodiment of the present disclosure. The asymmetric hair follicle spheroid 200 according to another embodiment of the present disclosure is patterned in the form of a hair follicle-mimicking structure, and has a spherical shape or a shape similar to the spherical shape. Here, the patterning may be achieved by positioning the partitioning member 110 partitioned in the form of a hair follicle-mimicking structure in the bio-ink accommodating part 130, supplying bio-ink containing different biological cells to each partitioned space of the partitioning member 110, and then discharging it through a nozzle.

The phrase “the asymmetric hair follicle spheroid 200 have asymmetry” means that the cross section of the hair follicle spheroid 200 has a structure in which it is asymmetric with respect to the X-axis and is symmetric with respect to the Y-axis, where the cross section is a cross section of a surface perpendicular to a direction in which the hair follicle spheroid 200 is discharged.

The asymmetric hair follicle spheroid 200 is formed from a bio-ink containing hydrogel and biological cells, and different bio-inks are filled into each partitioned space and then discharged, so that each region is formed from a bio-ink containing different biological cells.

The cross section of the asymmetric hair follicle spheroid 200 includes a first region 210 formed by discharging the first bio-ink, a second region 220 formed by discharging the second bio-ink, and a third region 230 formed by discharging the third bio-ink.

In the cross section of the asymmetric hair follicle spheroid 200, the first region 210 is formed to extend from a lower portion of the hair follicle spheroid to a central portion of the hair follicle spheroid along the Y-axis, the second region 220 has a half-donut shape, and a concave portion formed to surround an upper portion of the first region 210, and the third region 230 is positioned on the side surface of the first region 210 such that one side surface is formed by the first region 210 and an upper surface is formed by the second area 220.

Thus, an upper portion of the first region 210 is formed to be surrounded by the second region 220, and the remaining side portion of the first region 210 is formed to be surrounded by the third region 230.

By having such a shape, the directionality of the hair formed from the asymmetric hair follicle spheroid 200 may be controlled, and the shape and size uniformity of each asymmetric hair follicle spheroid 200 may be secured.

Hereinafter, the specific operations and effects of the present disclosure will be described with reference to an embodiment of the present disclosure. However, it is presented as a preferred example of the present disclosure, and the scope of rights of the present disclosure is not limited by the embodiment.

### Preparation Example 1

First, 4.5 w/v % of an initial collagen was neutralized to a pH of 7.1 to 7.5, and then the fibroblasts 3×10⁷ cells/ml, which are cells corresponding to dermal papilla (DP) cells, were stained with host 33342 (blue) to prepare first bio-ink. 4.5 w/v % of collagen was neutralized to pH of 7.1 to 7.5, and then HaCaT cells 3×10⁷ cells/ml, which are cells corresponding to outer root sheath (ORS) cells, were stained with CMFDA (green) to prepare second bio-ink. 4.5 w/v % of collagen was neutralized to pH of 7.1 to 7.5, and then EA.hy.926 3×10⁷ cells/ml, which are cells corresponding to vascular endothelial (Endo) cells, were stained with CMTPX (red) to prepare third bio-ink. Then, mineral oil was prepared.

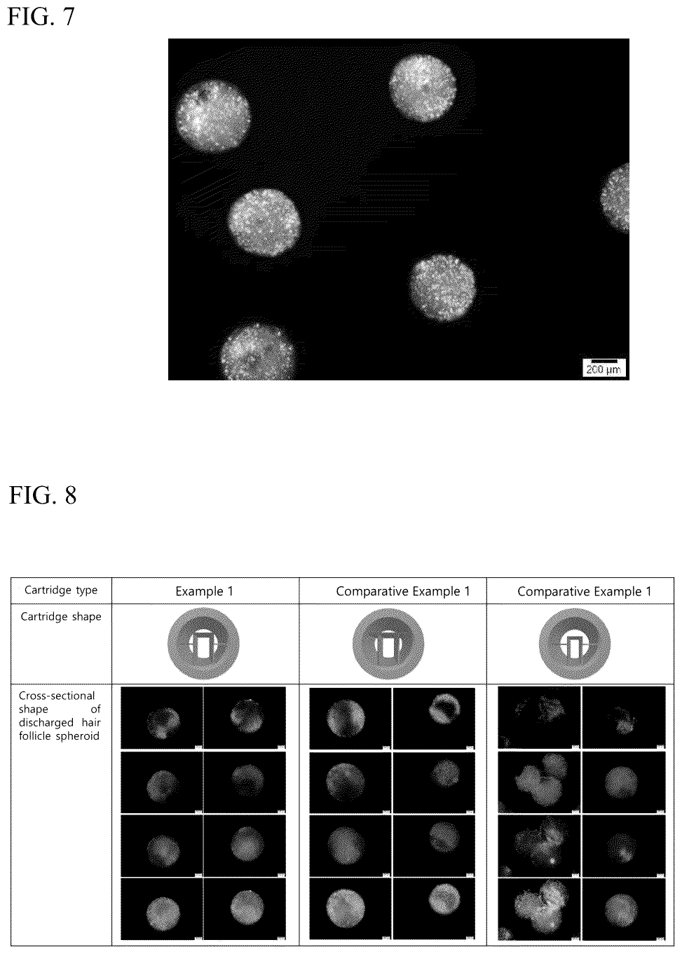

Next, a cartridge was prepared in which the first bio-ink was filled in the first partitioned space 111, the second bio-ink was filled in the second partitioned space 112, and the third bio-ink was filled in the third partitioned space 113 in a partitioning member having a cross-sectional shape illustrated in FIG. 2. A microfluidic device having a co-axial nozzle illustrated in FIG. 5 was prepared, the cartridge was mounted on the micro-fluidic device, and then, the hair follicle spheroid was prepared by allowing the hydrophobic solution to be continuously discharged from the outer nozzle 170 and the first, second, and third bio-inks to be discharged together but intermittently from the inner nozzle 161, which was observed using a fluorescence microscope and illustrated in FIG. 7.

Referring to FIG. 7, it can be confirmed that a hair follicle spheroid mimic was formed in a structure in which one end of the first bio-ink, which appears in blue, is surrounded by the second bio-ink, which appears in green, and the third bio-ink, which appears in red, is placed on both sides of the first bio-ink. Therefore, it could be confirmed that a spherical hair follicle spheroid with an asymmetric cross section can be produced using this experimental method, and the shape and size of each hair follicle spheroid were uniformly formed.

### Preparation Example 2

First, first bio-ink was prepared by using 4.5 w/v % of an initial collagen, adjusting a pH to 7.1 to 7.5, and adding blue fluorescent particles thereto. Second bio-ink was prepared by using 4.5 w/v % of collagen, adjusting a pH to 7.1 to 7.5, and then adding green fluorescent particles thereto. Third bio-ink was prepared by using 4.5 w/v % of the collagen, adjusting a pH to 7.1 to 7.5, and then adding red fluorescent particles thereto. Then, mineral oil was prepared. Next, a cartridge was prepared in which the first bio-ink was filled in the first partitioned space 111, the second bio-ink was filled in the second partitioned space 112, and the third bio-ink was filled in the third partitioned space 113 in a partitioning member having a cross-sectional shape illustrated in FIG. 2. A microfluidic device having a co-axial nozzle illustrated in FIG. 5 was prepared, the cartridge was mounted on the micro-fluidic device, and then, the hair follicle spheroid was prepared by allowing the hydrophobic solution to be continuously discharged from the outer nozzle 170 and the first, second, and third bio-inks to be discharged together but intermittently from the inner nozzle 161.

### Experimental Example

As illustrated in FIG. 8, partitioning members having three types of partitioned forms were prepared to produce a hair follicle spheroid in the same manner as in Preparation Example 2. In Example 1, a partitioning member having a shape according to an embodiment of the present disclosure was prepared. In Comparative Example 1, a partitioning member having a shape in which the upper portion of the first partitioned space 111 is not surrounded by the second partitioned space 112 was prepared. In Comparative Example 2, a partitioning member having a width of the first partitioned space 111 formed smaller than the width of each of the third partitioned spaces 113 on the X-axis was prepared.

Afterwards, each hair follicle spheroid was observed using a fluorescence microscope and illustrated together in FIG. 8.

Referring to the experimental results of FIG. 8, in the case of Example 1, it can be confirmed that the upper portion of the first region indicated in blue is surrounded by the second region indicated in green, and a third region indicated in red exists on the remaining side surface of the first region. It is expected that when a portion of the first region is surrounded by the second region, hair is produced and grows in the direction from a first region containing dermal papilla cells to a second region containing outer root sheath cells, and thus it is possible to control the direction of the hair when it has such a shape.

In the case of Comparative Example 1, the positions of the first, second, and third regions were similar to those of Example 1, but a part of the first region was formed such that no part of the first region was surrounded by the second region. It is determined that when no part of the first region was surrounded the second region, the direction of hair production cannot not be controlled.

In the case of Comparative Example 2, it seems that the first region was formed so minimally that it was not easily observed, so that a part of the first region was not surrounded by the second region, and therefore, the direction of hair production cannot not be controlled.

Therefore, it was confirmed from the results of this experiment that when a hair follicle spheroid was produced using a partitioning member having a cross-sectional shape of the partitioning member illustrated in FIG. 2 and in which the width of the first partitioned space 111 is formed smaller than the width of each of the third partitioned space 113 on the X-axis, the hair follicle spheroid was produced in a form in which the direction of hair production can be controlled.

The present disclosure is not limited to the specified embodiments and descriptions described above, and various modifications can be made by anyone having ordinary knowledge in the art in the art to which the present disclosure belongs without departing from the spirit of the present disclosure as claimed in the claims, and such modifications will fall within the scope of protection of the present disclosure.

### INDUSTRIAL APPLICABILITY

The present disclosure provides an asymmetric hair follicle spheroid by filling a plurality of bio-inks containing outer root sheath cells, dermal papilla cells, and inner root sheath cells into a partitioning member having regions partitioned in an asymmetric shape, respectively, and performing printing. Accordingly, it is possible to impart directionality so that hair grows in a specific direction, and individual hair follicle spheroids are produced by a bio-printing method, and thus, are simply and fast produced and have a uniform quality. Therefore, the present disclosure has an industrial applicability.

1. A method for producing an asymmetric hair follicle spheroid, the method comprising:
an accommodating part preparation step of preparing a bio-ink accommodating part 130;
a loading step of positioning a partitioning member 110 in which a plurality of partitioned spaces are formed, in the bio-ink accommodating part 130;
a bio-ink supplying step of supplying bio-ink containing hydrogel to each partitioned space of the partitioning member 110; and
a discharge step of applying pressure to the bio-ink accommodating part 130 and discharging the bio-ink accommodated in the partitioned space through a single nozzle,
wherein the partitioning member 110 in which the plurality of partitioned spaces are formed, includes the plurality of partitioned spaces including:
a first partitioned space 111 extending from a lower portion of the partitioning member 110 to a middle upper portion of the partitioning member 110 along the Y-axis of the partitioning member 110; a second partitioned space 112 formed on an upper portion of the partitioning member 110 so as to surround the upper portion of the first partitioned space 111 and having a half-donut shape; and a plurality of third partitioned spaces 113 having one side surface formed by the first partitioned space 111 and an upper surface formed by the second partitioned space 112, and
wherein a cross section of the partitioning member 110 in which the plurality of partitioned spaces are formed, has a structure in which it is asymmetric with respect to an X-axis of the partitioning member 110 passing through a center of the cross section, and is symmetric with respect to a Y-axis of the partitioning member 110 passing through the center of the cross section.

2. The method of claim 1, wherein first bio-ink containing dermal papilla cells is supplied to the first partitioned space 111,
second bio-ink containing outer root sheath cells is supplied to the second partitioned space 112, and
third bio-ink containing vascular endothelial cells is supplied to the third second partitioned space 113.

3. The method of claim 1, wherein the discharge step is performed simultaneously with the bio-ink supplying step or performed sequentially after the bio-ink supplying step.

4. An asymmetric hair follicle spheroid produced by the method of claim 1, and having a spherical shape or a shape similar to the spherical shape,
wherein a cross section of the hair follicle spheroid is asymmetric with respect to an X-axis and is symmetric with respect to a Y-axis, and
wherein the cross section of the hair follicle spheroid includes:
a first region 210 formed to extend from a lower portion of the hair follicle spheroid to a central portion of the hair follicle spheroid along the Y-axis; a second region 220 having a half-donut shape and having a concave portion formed to surround an upper portion of the first region 210; and a third region 230 formed to surround the remaining side surfaces of the first region 210.
